SEQUENCE LISTING (16 sequences)

SEQ ID NO: 1 - DNA, 18 bp, Artificial Sequence
  18-nt primer hybridization site
ataccagcttattcaatt

SEQ ID NO: 2 - DNA, 18 bp, Artificial Sequence
  18-nt primer hybridization site
agatagtaagtgcaatct

SEQ ID NO: 3 - DNA, 93 bp, Artificial Sequence
  Chemically synthesized sgc3 primer
ataccagcttattcaattcctgtgggaaggctatagaggggccagtctatgaataagatggcggactaatgtgtaagatagtaagtgcaatct

SEQ ID NO: 4 - DNA, 51 bp, Artificial Sequence
  Chemically synthesized sgc3b primer
acttattcaattcctgtgggaaggctatagaggggccagtctatgaataag

SEQ ID NO: 5 - DNA, 90 bp, Artificial Sequence
  Chemically synthesized sgc4 primer
ataccagcttattcaattcgagtgcggatgcaaacgccagacagggggacaggagataagaatagcgtgatgagatagtaagtgcaatct

SEQ ID NO: 6 - DNA, 71 bp, Artificial Sequence
  Chemically synthesized Sgc4a primer
ataccagcttattcaattcgagtgcggatgcaaacgccagacagggggacaggagatagtaagtgcaatct

SEQ ID NO: 7 - DNA, 56 bp, Artificial Sequence
  Chemically synthesized sgc4f primer
atcacttataacgagtgcggatgcaaacgccagacagggggacaggagataagtga

SEQ ID NO: 8 - DNA, 80 bp, Artificial Sequence
  Chemically synthesized sgc5 primer
ataccagcttattcaattaccgacgacgaactatctatcactatcttacacatcatacctcgagatagtaagtgcaatct

SEQ ID NO: 9 - DNA, 88 bp, Artificial Sequence
  Chemically synthesized sgc6 primer
ataccagcttattcaattcctgtgggaaggctatagaggggccagtctatgaacaagatggttgatccgtagatagtaagtgcaatct

SEQ ID NO: 10 - DNA, 87 bp, Artificial Sequence
  Chemically synthesized sgc7 primer
ataccagcttattcaattaccgcagcgactatctcgactacattactagcttatactccgatcatctctagatagtaagtgcaatct

SEQ ID NO: 11 - DNA, 88 bp, Artificial Sequence
  Chemically synthesized sgc8 primer
ataccagcttattcaattagtcacacttagagttctaactgctgcgccgccgggaaaatactgtacggttagatagtaagtgcaatct

SEQ ID NO: 12 - DNA, 41 bp, Artificial Sequence
  Chemically synthesized sgc8c primer
atctaactgctgcgccgccgggaaaatactgtacggttaga

SEQ ID NO: 13 - DNA, 88 bp, Artificial Sequence
  Chemically synthesized sga16 primer
ataccagcttattcaattagtcacacttagagttctagctgctgcgccgccgggaaaatactgtacggatagatagtaagtgcaatct

SEQ ID NO: 14 - DNA, 18 bp, Artificial Sequence
  5′-primer
ataccagcttattcaatt

SEQ ID NO: 15 - DNA, 18 bp, Artificial Sequence
  5′-primer
ataccagcttattcaatt

SEQ ID NO: 16 - DNA, 18 bp, Artificial Sequence
  3′-primer
agattgcacttactatct

(19) United States
(12) Patent Application Publication
(10) Pub. No.: US 2009/0130650 A1
(43) Pub. Date: May 21, 2009

(54) Methods for the production of highly sensitive and specific cell surface probes

(76) Inventors: Weihong Tan, Gainesville, FL (US); Dihua Shangguan, Beijing (CN); Ying Li, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/708,760

(22) Filed: Feb. 20, 2007

Related U.S. Application Data
(60) Provisional application No. 60/774,949, filed on Feb. 17, 2006.
(60) Provisional application No. 60/780,332, filed on Mar. 8, 2006.

(51) Int. Cl. C12Q 1/70; C12Q 1/68; C07H 21/04

(52) U.S. Cl. 435/5; 435/6; 536/24.31

(57) ABSTRACT

A system and method for producing an oligonucleotide having a high affinity for extracellular or cell surface markers on a target cell. The resultant oligonucleotide probe can be used to detect a target biomolecule, in particular a cancer cell or infectious agent such as a bacterium, virus, or fungus, comprising an aptamer having a high affinity for the biomolecule, wherein at least one labeled dye is attached to the aptamer. The labeled dye causes the aptamer to emit a baseline, non-visible emission. When the aptamer (also referred to herein as a probe) of the invention interacts with a target biomolecule, the fluorescence emission changes from the baseline emission to an emission that is visually detectable.

### CROSS-REFERENCE TO A RELATED APPLICATION

This application claims the benefit of U.S. provisional application Ser. Nos. 60/774,949, filed on Feb. 17, 2006 and 60/780,332, filed on Mar. 8, 2006, both of which are hereby incorporated by reference in their entirety, including all figures, tables, and drawings.

### GOVERNMENT SUPPORT

The subject matter of this application has been supported in part by U.S. Government Support under NIH GM66137; NIH NS045174; and NSF EF0304569. Accordingly, the U.S. Government has certain rights in this invention.

### FIELD OF THE INVENTION

The present invention provides a novel molecular probe and method for synthesizing the probe, which has the ability to rapidly bind to a cancer biomarker protein either in vivo or in vitro with a high degree of sensitivity and selectivity, whereupon binding of the probe to the protein produces a detectable signal for use in medical diagnosis.

### BACKGROUND OF THE INVENTION

Most cancers are diagnosed based on morphologic features of tumor tissues or cells. These morphologic features cannot reliably be related to the complicated molecular events underlying neoplastic processes (Luo, J. et al., “Looking beyond morphology: cancer gene expression profiling using DNA microarrays,” Cancer Invest, 21(6):937-949 (2003)). Molecular profiling (MP) identifies molecular signatures (biomarkers) that are associated with diseases such as cancer (see, for example, Dhanasekaran, S. M. et al., “Delineation of prognostic biomarkers in prostate cancer,” Nature, 412(6849):822-6 (2001); and Sander, C., “Genomic medicine and the future of health care,” Science, 287(5460):1977-78 (2000)). Various types of cancers can behave very differently because of diverse underlying molecular aberrations (Espina, V. et al., “Pathology of the future: molecular profiling for targeted therapy,” Cancer Invest., 23(1):36-46 (2005)).

There is a need for tools that provide accurate and efficient molecular analysis to aid in characterizing tumors by their molecular signatures. Currently, there are very few biomarkers known for use in effectively distinguishing tumor cells from their normal cell counterparts (Sternberg, S. S. et al., Diagnostic Surgical Pathology, Lippincott Williams and Wilkins, 3^{rd} ed., 1999). One approach for identifying biomarkers is to develop molecular probes that recognize cell surface markers with high affinity and specificity. Aptamers, single-stranded DNA (ssDNA), RNA, or modified nucleic acids, are good MP candidates. They have the ability to bind specifically to targets, which range from small organic molecules to proteins (see, for example, Osborne, S. E. and A. D. Ellington, “Nucleic Acid Selection and the Challenge of Combinatorial Chemistry,” Chem. Rev., 97(2):349-370 (1997); Nutiu, R. and Y. Li, “In vitro selection of structure-switching signaling aptamers,” Angew Chem Int Ed Eng., 44(7):1061-5 (2005); and Wilson, D. S, and J. W. Szostak, “In vitro selection of functional nucleic acids,” Annu Rev Biochem, 68:611-47 (1999)).

The basis for target recognition is the tertiary structures formed by the single-stranded oligonucleotides (Breaker, R. R., “Natural and engineered nucleic acids as tools to explore biology,” Nature, 432(7019):838-45 (2004)). These aptamers are obtained through an in vitro selection process known as SELEX (Systematic Evolution of Ligands by Exponential Enrichment) (see Ellington, A. D. and J. W. Szostak, “In vitro selection of RNA molecules that bind specific ligands,” Nature, 346(6287):816-22 (1990); and Tuerk, C. and L. Gold, “Systematic evolution of ligands by exponential enrichment: RNA ligands to bacteriophage T4 DNA polymerase,” Science, 249(4968):505-10 (1990)), by which the aptamers are selected from libraries of random sequences of synthetic DNA or RNA by repetitive binding of these oligonucleotides to target molecules.

Most of the aptamers reported so far have been selected using one type of molecules, such as purified proteins. The aptamer-selection against complex targets (such as red blood cell membranes and endothelial cells) has also been demonstrated (see, for example, Morris, K. N. et al., “High affinity ligands from in vitro selection: complex targets,” Proc. Natl. Acad. Sci. USA., 95(6):2902-7 (1998); and Blank, M. et al., “Systematic evolution of a DNA aptamer binding to rat brain tumor microvessels. selective targeting of endothelial regulatory protein pigpen,” J. Biol. Chem., 276(19):16464-8 (2001)). To date, the cell-based SELEX process has not been used for selecting a panel of tumor-specific aptamers for molecular profiling of cancers cells and for use in disease biomarker discovery.

### BRIEF SUMMARY OF THE INVENTION

The present invention provides systems and methods for detecting biomolecules either in vitro or in vivo for clinical diagnosis. In one embodiment, the present invention provides novel methods for preparing aptamers having an affinity for target biomolecules present on a target cell (such as specific markers on cancer cells), without prior knowledge of the target biomolecules located on the target cell.

In one embodiment, the present invention provides an efficient high-throughput system for the molecular analysis of cells, leading to the identification of novel peptides (aptamers) that function extracellularly and/or intracellularly. Thus, the present invention surpasses existing research strategies that rely on targeted identification and selection, including those based on elucidation of specific protein-protein interactions, phenotypic gene expression profiling, or genotypic analysis. This is especially advantageous in the study of a complex and highly diverse disease such as cancer.

One aspect of the invention relates to the development of oligonucleotide probes for diagnostic and therapeutic applications for infectious diseases and emerging pathogens. Molecular level differences on the surface (extracellular) and within (intracellular) diseased and healthy cells are exploited in accordance with the methods disclosed herein to generate nucleic acid probes (aptamers) specific for a target infectious disease or pathogen. Thus, pathogenic virus, bacteria, fungi and cells infected with virus, bacteria, fungus, etc. are employed to identify and derive high affinity aptamers. Such probes are highly specific and can be used as biosensors and molecular probes for detection of biowarfare agents, for the early detection of infectious disease, and have the potential to prevent or reverse pathological conditions caused by an infectious agent.

According to the subject invention, identification of specific molecular signatures on the cancer cell surface enables definition of tumors of other diseases as entities that are biologically homogeneous. Moreover, the molecular characteristics of a specific tumor can be used in accordance with the invention to develop tailored treatment regimes, to monitor therapeutic responses, and to detect residual diseases.

In a preferred embodiment, the cell-based SELEX process of the invention (see, for example FIGS. 1A and 1B) uses whole cells as targets to select aptamers that can recognize target cells. A group of cell-specific aptamers can be selected using a subtraction strategy without knowing the target molecules present on the cell surface. Not only can the selected aptamers be used as biomarkers for molecular profiling of disease, but they can also be used as tools for identifying new biomarkers of diseased cells.

In one embodiment of the invention, cultured leukemia cells were used as targets for aptamer selection because they are homogeneous and their surface properties can be characterized using known molecular profiles. In addition, flow cytometry analysis can be used to effectively monitor the selection process and to evaluate the selected aptamers.

Types of leukemia cell lines that are differentiated using aptamers of the invention include, but are not limited to, acute lymphoblastic leukemia cells; T-cell lymphoblasts (such as MOLT-4 and CCRF-CEM); and B-cell lymphoblasts (such as SUP-B15).

In a related embodiment, the precursor T cell acute lymphoblastic leukemia cell line, CCRF-CEM (CEM), was used for cell-specific aptamer selection, and a B cell lymphoma cell line (Ramos) was used as the negative control for counter selection. A negative selection step is necessary due to the commonality of many surface molecules for both the CEM and Ramos cells. To select the appropriate aptamer, a nucleic acid sample, such as an ssDNA library containing 52-mer random DNA sequences flanked by two 18-mer PCR primer sequences, was used. The progress of the selection process was monitored using flow cytometry.

In one embodiment, an increased number of selection cycles is utilized to enrich and identify DNA sequences with better binding affinity to the target cells. This was confirmed via observed steady increases in fluorescence intensity in CEM cells (target cells bound with fluorophore-labeled selected DNA sequences) in flow cytometry analysis. There was no significant change in fluorescence intensity on Ramos cells (also referred to herein as control or counter-selective cells). These results indicate that DNA probes that specifically recognize surface biomarkers on CEM cells were selected (FIG. 2). The specific binding of the selected pools of DNA probes (aptamers) to the target cells was further confirmed by confocal microscopy imaging (FIG. 4). After incubation with the fluorophore-labeled selected aptamer pool, the CEM cells showed very bright fluorescence on the periphery of cells, while the Ramos cells displayed no significant fluorescence.

Accordingly, it is an object of the invention to develop aptamers having the ability to differentiate leukemia cell lines because such aptamers are important in medical diagnostics; many of the leukemia cell membrane markers and receptors are well understood; and because there is an easy choice in different types of leukemia cells for use in control experiments, in both the selection and application of aptamers.

It is another object of the invention to develop therapeutic aptamers or aptamer-conjugated drugs for targeting specific tumor cells in personalized therapeutic medicine regimes.

Another objective of the subject invention is the real-time monitoring and quantitation of intracellular molecules (such as genes and proteins) in living cells and tissues.

The aptamer of the invention can be molecularly engineered to have a high affinity for compounds other than biomolecules, such as nucleic acids or toxic substances. Labeled dyes such as pyrene are then attached to either end of the aptamer to form a probe of the invention. Such highly sensitive probes are especially advantageous for use in clinical, forensic, and environmental applications.

### BRIEF DESCRIPTION OF THE FIGURES

The file of this patent contains at least one drawing executed in color. Copies of this patent with color drawings(s) will be provided by the Patent and Trademark Office upon request and payment of the necessary fee.

FIGS. 1A and 1B illustrate schematic presentations of the cell-based aptamer selection process (also referred to herein as Cell-Selex) in accordance with the subject invention.

FIG. 2 is a flow cytometry assay for the binding of selected pool with CCRF-CEM cells (target cells) and Ramos cells (negative/control cells). The green curve represents the background binding of the unselected DNA library.

FIGS. 3A, B, and C are graphical illustrations characterizing the selected aptamers sga16 (SEQ ID NO. 12) and sgc8 (SEQ ID NO. 10).

FIG. 4 is a confocal image of cells stained by the 20^{th} round selected pool labeled with TMR. The top left panel is a fluorescent image of CCRF-CEM cells (target cells); the top right panel is an optical image of CCRF-CEM cells. The bottom left panel is a fluorescent image of Ramos cells (control cells). The bottom right panel is an optical image of Ramos cells.

FIGS. 5A and B are graphical illustrations an aptamer of the invention (sgc3, SEQ ID NO. 2) and their ability to recognize a subset of target CCRF-CEM cells.

FIG. 6 is an image of a flow cytometry assay for the binding of sequence sga4 with CCRF-CEM cells and Ramos cells. The final concentration of these sequences in binding buffer was 0.5 μM.

FIGS. 7A and B are graphical illustrations flow cytometry analyses of CEM cells and human bone marrow cells incubated with FITC-labeled sgc8 (SEQ ID NO. 10), and PE-labeled anti-CD3 antibody, and PerCP-labeled anti-CD45 antibody.

FIG. 8 provides flow cytometry analyses of CCRF-CEM cells and human bone marrow cells labeled with FITC-labeled sgc3 (SEQ ID NO. 2), PE-labeled anti-CD3 antibody, and PerCP-labeled anti CD45 antibody.

FIG. 9 provides flow cytometry analyses of CCRF-CEM cells and human bone marrow cells labeled with sgc4 (SEQ ID NO. 4).

FIG. 10 provides a schematic description of the enrichment of target aptamers in accordance with the present invention.

FIG. 11 provides graphically illustrated results from flow cytometry monitoring of the enrichment of aptamers of FIG. 10.

FIG. 12 provides a graphically illustrated result of a flow cytometry assay of the aptamers synthesized and selected as illustrated in FIG. 10.

FIG. 13 provides graphically illustrated results from flow cytometry assays of the aptamers synthesized and selected as illustrated in FIG. 10 against target cells.

FIG. 14 provides confocal images of target cells incubated with certain aptamers prepared and selected in accordance with the schemes illustrated in FIG. 10.

FIG. 15 is a table listing the characteristics of various probes obtained using the methods described herein.

### BRIEF DESCRIPTION OF THE SEQUENCE LISTING

SEQ ID NO.1 is single stranded DNA having a central randomized sequence of 52 nucleotides (nt) flanked by 18-nt primer hybridization sites that is used for obtaining a probe of the invention.

SEQ ID NOS. 2-12 are nucleotide sequences of probes obtained using the methods of the invention.

SEQ ID NO:13 is the nucleotide sequence of a fluorescein isothiocyanate (FITC)-labeled 5′-primer.

SEQ ID NO:14 is the nucleotide sequence of a tetramethylrhodamine (TMRA)-labeled 5′-primer.

SEQ ID NO:15 is the nucleotide sequence of a triple biotinylated (trB) 3′-primer.

### DETAILED DESCRIPTION OF THE INVENTION

The present invention provides systems and methods for detecting biomolecules either in vitro or in vivo for clinical diagnosis. In one embodiment, the present invention provides novel methods for preparing aptamers having an affinity for target biomolecules present on a target cell (e.g., specific markers on extracellular or intracellular diseased and healthy cells, such as cancer cells or pathogenic, virus, bacteria, fungi and cells infected with virus, bacteria, and fungus), without prior knowledge of the target biomolecules located on the target cell.

In one embodiment, the present invention provides an efficient high-throughput system for the molecular analysis of cells, leading to the identification of novel peptides (aptamers) that function extracellularly and/or intracellularly. Thus, the present invention surpasses existing research strategies that rely on targeted identification and selection, including those based on elucidation of specific protein-protein interactions, phenotypic gene expression profiling, or genotypic analysis. This is especially advantageous in the study of a complex and highly diverse disease such as cancer or infectious diseases (such as those associated with virus, bacteria, and fungi).

The target cells may be whole organisms such as bacterium, virus, or single-celled protozoan pathogens; or they may be biological cells such as cancer cells. The target cells may be present in samples of animal tissue, biological fluid, or environmental substances such as plant material, water, beverages, and industrial waste.

In one embodiment, the subject invention provides a probe comprising a molecularly engineered aptamer having a high affinity for a target compound (such as a leukemia cell; CEM), wherein at least one labeled dye is attached to the aptamer. When unbound, the probes of the invention emit a baseline emission. Once a probe of the invention binds to a target compound, the labeled dye emits a detectable second emission, different from that of the baseline emission.

The probe of the invention are derived from aptamers, which have the capacity for forming specific binding pairs with virtually any chemical compound, whether monomeric or polymeric. One procedure for the selection of aptamers that bind to a desired target compound in accordance with the present invention is known as SELEX. SELEX is the in vitro evolution of nucleic acid molecules having highly specific binding ability to target molecules and is described in U.S. patent application Ser. No. 07/536,428 entitled “Systematic Evolution of Ligands by Exponential Enrichment,” now abandoned; U.S. Pat. No. 5,475,096 entitled “Nucleic Acid Ligands”; and U.S. Pat. No. 5,270,163 entitled “Methods of Identifying Nucleic Acid Ligands” (see also WO 91/19813), each of which is specifically incorporated by reference herein. Each of these references describes a fundamentally novel method for making an aptamer to any desired target molecule.

In accordance with the subject invention, SELEX-like processes, such as those disclosed in U.S. patent application Ser. No. 07/960,093 entitled “Method for Selecting Nucleic Acids on the Basis of Structure,” can be used to prepare aptamers of the invention. The SELEX-like process of the '093 application enables the selection of nucleic acid molecules with specific structural characteristics, such as bent DNA. Other disclosed SELEX-like processes that can be used according to the subject invention include, but are not limited to, the following: U.S. patent application Ser. No. 08/123,935 entitled “Photoselection of Nucleic Acid Ligands,” which describes a SELEX-based method for selecting nucleic acid ligands containing photoreactive groups capable of binding and/or photocrosslinking to and/or photoinactivating a target molecule; U.S. Pat. No. 5,580,737 entitled “High-Affinity Nucleic Acid Ligands That Discriminate Between Theophylline and Caffeine,” which describes a method for identifying highly specific nucleic acid ligands able to discriminate between closely related molecules, which can be non-peptidic, termed Counter-SELEX; and U.S. Pat. No. 5,567,588 entitled “Systematic Evolution of Ligands by Exponential Enrichment: Solution SELEX,” which describes a SELEX-based method which achieves highly efficient partitioning between oligonucleotides having high and low affinity for a target molecule.

Aptamers with improved characteristics (such as improved in vivo stability or improved delivery characteristics) can be prepared using techniques that are known to those of ordinary skill in the art. For example, chemical substitutions at the ribose and/or phosphate and/or base positions can be performed to improve aptamer stability in vivo. Additional techniques for improving aptamer characteristics include those described in U.S. Pat. No. 5,660,985 entitled “High Affinity Nucleic Acid Ligands Containing Modified Nucleotides,” that describes oligonucleotides containing nucleotide derivatives chemically modified at the 5- and 2′-positions of pyrimidines; U.S. patent application Ser. No. 08/134,028, which describes highly specific Nucleic Acid Ligands containing one or more nucleotides modified with 2′-amino (2′-NH.sub.2), 2′-fluoro (2′-F), and/or 2′-O-methyl (2′-OMe); and U.S. patent application Ser. No. 08/264,029 entitled “Novel Method of Preparation of Known and Novel 2′ Modified Nucleosides by Intramolecular Nucleophilic Displacement,” which describes oligonucleotides containing various 2′-modified pyrimidines.

The subject invention provides another approach, the cell-SELEX approach, for identifying and isolating tumor-specific aptamers that are extremely useful in molecular profiling of targeted or diseased cells. Such a selection does not require prior knowledge of biomarker targets. The selection process is simple, reproducible, and straightforward. The aptamers of the invention can bind to target cells with Kd in the nM to pM range. Using the selected aptamers of the invention as molecular profilers for molecular profiling of cancer cells has yielded interesting information (such as regarding leukemia cells and normal human bone marrow aspirate). For example, some of the subject aptamers can only recognize a subset of the target cells, while others can bind to only one or two types of cancer cells. In addition, the isolation and identification of the target molecules recognized by these selected aptamers provide an effective and rapid way to discover disease biomarkers.

The disclosed method is particularly advantageous in that the subject probes can be prepared in large scale easily as well as being relatively inexpensive to produce and stable. Such probes are highly effective for identifying drug resistant organisms in the event that probes against a parental organism fail to recognize a newly resistant strain.

Detectable agents (such as labeled dyes) can be attached to an aptamer to form a probe of the invention. The labeled dyes of the invention can be selected from many reactive fluorescent molecules that are known by and readily available to those of skill in the art. Specific labeled dyes that are useful in practicing the subject invention include, but are not limited to, dansyl, fluorescein, 8-anilino-1-napthalene sulfonate, pyrene, ethenoadenosine, ethidium bromide prollavine monosemicarbazide, p-terphenyl, 2,5-diphenyl-1,3,4-oxadiazole, 2,5-diphenyloxazole, p-bis[2-(5-phenyloxazolyl)]benzene, 1,4-bis-2-(4-methyl-5-phenyloxazolyl)benzene, and lanthanide chelate. Preferably, the probes of the invention use pyrene.

In certain embodiments, moieties such as enzymes, or other reagents, or pairs of reagents, that are sensitive to the conformational change of an aptamer binding to a target molecule, are incorporated into the engineered aptamers to form the aptamer probes. Such moieties can be incorporated into the aptamer either prior to transcription or post-transcriptionally, and can potentially be introduced either into known aptamers or into a pool of oligonucleotides from which the desired aptamers are be selected.

Upon binding of the aptamer probe to a target molecule, such moieties are activated and generate concomitant signals (for example, in the case of a fluorescent dye an alteration in fluorescence intensity, anisotropy, wavelength, or FRET). Such probes are particularly useful for clinical diagnosis of diseases (such as infections caused by organisms or cancer cells).

In other embodiments of the invention, moieties such as radioactive compounds or other known therapeutic compounds can be bound to the aptamer probe so as to provide treatment for the diseased cell. For example, a radioactive compound can be bound to an aptamer probe of the invention to act as an anti-bacterial, anti-viral and/or anti-fungal agent.

In yet another aspect, the invention features a method or system for simultaneously detecting the presence or absence of one or more different target molecules in a sample using a plurality of different species of aptamer probes, wherein each species of aptamer probes has a different moiety or label dye group, a binding region that binds to a specific non-nucleic acid target molecule, and wherein the binding regions of different aptamers bind to different target molecules; and a detection system that detects the presence of target molecules bound to aptamer probes, the detection system being able to detect the different moiety or label dye groups.

The method can also be carried out with a plurality of identical aptamer probes. For example, each aptamer can include a moiety such as a molecular beacon that changes fluorescence properties upon target binding. Each species of aptamer probe can be labeled with a different fluorescent dye to allow simultaneous detection of multiple target molecules, e.g., one species might be labeled with fluoroscein and another with rhodamine. The fluorescence excitation wavelength (or spectrum) can be varied and/or the emission spectrum can be observed to simultaneously detect the presence of multiple targets.

### Attachment of Aptamer Probes to a Solid Substrate

Solid supports for holding aptamer probes can be, e.g., a planar sheet of glass, such as a glass slide. Other solid surfaces are also suitable, such as metal, plastic, and ceramic.

An aptamer probe of the invention can be affixed to a glass slide by attaching an amine group of a quencher moiety of the aptamer probe to the glass via a linker molecule. First, a linker molecule is attached to the glass slide by dipping the slide into a solution including the linker and acidic water (pH 3.0) at a high temperature (such as 90° C.) for several hours. Afterwards, the linker molecule will coat the surface of the slide. Next, aptamer probes are attached to the linker molecules on the slide via the amine group of quencher moiety. To attach via the amine groups, the coated surface of the glass is exposed to a solution including the aptamer probe/quencher pair and CH₃CN at low temperatures (such as 20° C. for 1.5 hours). The aptamer probes can be localized to a particular spot on the glass slide by applying a microdrop of the aptamer probe-CH₃CN solution to a precise point on the slide using a robotic micropipetter. See, e.g., Schena et al., “Parallel Human Genome Analysis: Microarray-Based Expression Monitoring of 1000 Genes,” Proc. Nat'l Acad Sci. USA, 93: 10514-19 (1996).

As understood by the skilled artisan, an aptamer probe having an extended linker can be attached to the glass slide directly, rather than via a quencher. Various known linker molecules can be used. The extended linker can allow the aptamer probe to extend further into the liquid above the slide, facilitating binding of target molecules. The procedure for attaching the aptamer probe is similar to the procedure for attaching the aptamer probe/quencher.

Other methods for attaching oligonucleotides to glass are described in Shalon et al., “A DNA Microarray System for Analyzing Complex DNA Samples Using Two-Color Fluorescent Probe Hybridization,” Genome Res., 6:639-45 (1996) (oligonucleotides UV crosslinked to a poly-L-lysine coated surface), and Morgan and Taylor, “A Surface Plasmon Resonance Immunosensor Based on the Streptavidin-Biotin Complex,” Biosens. Biolectron., 7:405-10 (1992) (aptamers attached using streptavidin).

A variety of schemes to detect binding of aptamer probes to target molecules can be employed. First, fluorescent label dyes of the aptamer probes can be monitored, e.g., for changes in fluorescence efficiency. Second, changes in the Raman emission of the aptamer probes caused by the presence of a target molecule can be observed. Third, shifts in surface plasmon resonances at the surface of the array can be detected by monitoring the change in the wavelength or incident angle of absorbed light, or by using a Mach-Zehnder interferometer. Fourth, the detectable moieties can be enzymes or chemicals that can be monitored for changes in physical properties that occur when the aptamer probe changes conformation upon binding to a target molecule.

### Fluorescence Based Detection

To detect binding by monitoring fluorescence emission, fluorophores can be incorporated into the aptamer probes. These fluorophores are configured so that their fluorescence efficiency changes when a target molecule binds to the aptamer probe and changes the aptamer probe's conformation, thereby signaling the presence of target molecules in the sample. Fluorescence efficiency can be measured, e.g., using evanescent wave excitation and a cooled CCD camera or single-photon-counting detector.

### Fluorophore Reporter Moieties

Fluorophore reporter moities can be, e.g., a fluorescence energy transfer pair that signals a conformation change in an aptamer probe, or conventional fluorescent labels whose efficiency is dependent on the conformation of the aptamer probe.

Aptamer beacon reporter moieties can be a fluorophore and quencher or a charge or energy transfer system. A fluorophore can be 5-(2′-aminoethyl)aminoapthalene-1-sulfonic acid (“EDANS”), fluorescein, or anthranilamide. A quencher can be a chemical group, such as 4-(4′-dimethylaminophenylazo)benzoic acid (“DABCYL”), rhodamine, or eosine. A fluorophore and quencher can be incorporated into aptamer probes using techniques known in the art. See, e.g., Tyagi and Kramer, “Molecular Beacons: Probes That Fluoresce Upon Hybridization,” Nature Biotech., 14:303-08 (1996).

The detectable moiety groups can also include an energy transfer system. An aptamer probe has an oligonucleotide with a binding region configured to bind a target molecule. The detectable moiety group includes an acceptor/fluorescence emitting moiety and a donor/energy absorbing moiety attached to oligonucleotide. When the emitting moiety and absorbing moiety are in proximity, energy transfers between the moieties to emit fluoresces efficiently. A fluorescence emitting moiety can be Cy5. An absorbing moiety can be fluorescein or tetramethyl rhodamine (“TMR”). The emitting moiety and absorbing moiety can be attached to oligonucleotides of the aptamer probe using techniques known in the art. See, e.g., Sixou et al., “Intracellular Oligonucleotide Hybridization Detected by Fluorescence Resonance Energy Transfer (FRET),” Nucleic Acids Res., 22:662-68 (1994).

Instead of designing aptamer probes with energy transfer reporters, other fluorescence reporters known in the art can be used. For example, an aptamer probe can be labeled with a fluorophore whose fluorescence efficiency depends on the environment (such as electrical, physical, or chemical environment) of the molecule to which it is attached. For example, binding of the target molecule to the aptamer-probe changes the conformation of the aptamer probe, thereby changing the chemical environment of the fluorophore, thereby causing a detectable change in the fluorescence of the fluorophore.

Pyrene is a spatially sensitive fluorescent dye (see Fujimoto, K. et al., “Unambiguous detection of target DNAs by excimer-monomer switching molecular beacons,” Journal of Organic Chemistry, 69:3271-3275 (2004); Birks, J. B., Photophysics of Aromatic Molecules (Wiley Monographs in Chemical Physics) (1970); Winnik, F. M., “Photophysics of Preassociated Pyrenes in Aqueous Polymer-Solutions and in Other Organized Media,” Chemical Reviews, 93:587-614 (1993); and Lakowicz, J. R., Principles of Fluorescent Spectroscopy (Kluwer Academic/Plenum Publishers, New York, 1999)). Another example of a spatially sensitive fluorescent dye includes, but is not limited to, BODIPY Fl (see Dahim, M. et al., “Physical and photophysical characterization of a BODIPY phosphatidylcholine as a membrane probe,” Biophysical Journal, 83:1511-1524 (2002); and Pagano, R. E. et al., “A novel fluorescent ceramide analogue for studying membrane traffic in animal cells: accumulation at the Golgi apparatus results in altered spectral properties of the sphingolipid precursor,” Journal of Cell Biology, 113:1267-1279 (1991)). Both of these dyes, pyrene and BODIPY Fl, can form excited state dimers (excimers) upon close encounter of an excited state with another ground state molecule. The excimer emits at a longer wavelength than does a monomer.

An excimer is formed between two spatially sensitive fluorescent dyes (i.e., pyrenes) that are connected by a flexible covalent chain. As with FRET, the emission of the excimer is dependent upon the distance between the dyes. The stringent distance-dependent property of excimer formation is used in accordance with the subject invention as a unique means for signal transduction in the development of molecular probes. This is especially useful for developing aptamer probes due to the fact that many aptamers, like aptamers for PDGF-BB (see Fang, X. H., et al., “Molecular aptamer for real-time oncoprotein platelet-derived growth factor monitoring by fluorescence anisotropy,” Analytical Chemistry, 73:5752-5757 (2001); Nutiu, R. & Li, Y. F. “Structure-switching signaling aptamers: Transducing molecular recognition into fluorescence signaling,” Chemistry—A European Journal, 10:1868-1876 (2004); and Green, L. S. et al., “Inhibitory DNA ligands to platelet-derived growth factor B-chain,” Biochemistry, 35:14413-14424 (1996)), cocaine (Stojanovic, M. N. et al., “Aptamer-Based Folding Fluorescent Sensor for Cocaine,” Journal of the American Chemical Society, 123:4928-4931 (2001)), and thrombin (Paborsky, L. R. et al., “The single-stranded DNA aptamer-binding site of human thrombin,” Journal of biological chemistry, 268:20808-20811 (1993); and Hamaguchi, N. et al., “Aptamer beacons for the direct detection of proteins,” Analytical Biochemistry, 294:126-131 (2001)) undergo conformation change upon target binding.

The labeled dyes of the invention can be attached to any location on an aptamer of the invention, including sites on the base segment and sites on the sugar segment. In a preferred embodiment, the labeled dyes of the invention are attached to the terminal ends of the aptamer.

Many methods are available and appropriate for use in preparing the various labeled aptamers required to practice the present invention. One skilled in the art will be able, without undue experimentation, to choose a suitable method for preparing a desired fluorescently labeled aptamer. Additionally, as the art of organic synthesis, particularly in the area of nucleic acid chemistry, continues to expand in scope new methods will be developed which are equally as suitable as those now known.

In one embodiment, the aptamer of the invention is labeled by preparing, purifying, and characterizing a manifold of derivatized, labeled nucleic acids. For example, a labeled dye is attached to a nucleic acid sequence, which serves as a primer for nucleic acid synthesis. A nucleic acid polymer is then annealed to the primer nucleic acid sequence to form an aptamer of the invention.

Chemical methods are available to introduce fluorescence into specific nucleic acid bases by the reaction of chloracetaldehyde with adenosine and cytidine to give fluorescent products. The reaction can be controlled with respect to which of the two bases is derivatized by manipulating the pH of the reaction mixture; the reaction at 37° C. proceeds rapidly at the optimum pH of 4.5 for adenosine and 3.5 for cytidine. See Barrio et al., Biochem. Biophys. Res. Commun. 46:597-604 (1972). This reaction is also useful for rendering fluorescent the deoxyribosyl derivatives of these bases. See Kochetkov et al., Dokl. Akad. Nauk. SSSR C 213:1327-1330 (1973).

In addition to the various methods for converting the bases of an intact aptamer into their fluorescent analogs, there are a number of methods for introducing fluorescence into an aptamer during its de novo synthesis. For example, a fluorescently tagged linker can be used that tethers an oligonucleotide strand to a solid support. When the oligonucleotide strand is cleaved from the solid support, the fluorescent tether remains attached to the oligonucleotide. This method affords an aptamer that is fluorescently labeled at its 3′-end.

In a variation on this method, the 3′-end of the nucleic acid is labeled with a linker that bears an amine, or other reactive or masked reactive group, which can be coupled to a reactive fluorophore following cleavage of the oligonucleotide from the solid support. This method is particularly useful when the fluorophore is not stable to the cleavage or deprotection conditions.

Another method relies on the selective labeling of the 5′ terminus of the oligonucleotide chain. Although many methods are known for labeling the 5′ terminus, the most versatile methods make use of phosphoramidites, which are derivatized with fluorophore or, if the fluorophore is unstable under the cleaving and deprotection conditions, a protected reactive functional group. The reactive functional group is labeled with a fluorophore following cleavage and deprotection of the oligonucleotide and deprotection of the reactive functional group.

The probes of the invention have the ability to interact with any target compound or cell (such as virus, bacteria, fungus, cancer). Contemplated target compounds include, but are not limited to, small organic molecules (e.g., pesticides, herbicides, drugs, controlled substances, metabolites, explosive residues, plasticizers, industrial and agricultural pollutants, hormones); peptides and proteins (e.g., surface antigens on viruses, peptide hormones, cellular components); polysaccharides (e.g., surface antigens on bacteria and other pathogens); and other molecules (such as cancer cells, leukemia cells). In a preferred embodiment, the subject invention provides probes having a high affinity for cancer cells, in particular, acute lymphoblastic leukemia cells, T-cell lymphoblasts (such as MOLT-4 and CEM cancer cells), and B-cell lymphoblasts (such as SUP-B15 cells).

The subject invention utilizes the unique properties of aptamers to form probes for use in therapeutic practices, disease diagnosis and protein functional studies. These aptamers, which are integrated with a novel signal transduction mechanism, form sensitive and selective probes for use in protein detection. In one embodiment, the signal transduction mechanism is provided by spatially sensitive fluorescent dyes that form an excimer. The generation of the excimer emission requires the conformation change of the aptamer brought about by complexation with a target protein to bring two pyrene molecules together. This stringent requirement prevents false positive signals when the probe is digested by nucleases.

The probe of the invention is particularly useful in that it is able to detect protein in homogeneous solution and in real time. Another advantage of using the probe of the invention is that it allows ratiometric measurement, which could minimize the environmental effect to afford more precise detection. More importantly, excimer light switching approach significantly solves background signal problems both from the probe itself and other biological species.

Using the methods disclosed herein, a highly sensitive and selective aptamer probe can detect a target compound that is provided in pico-mole concentrations. For example, a CEM probe of the invention has demonstrated detection at very low concentrations of CEM. In addition, the visual detection of CEM is possible with the naked eye in a few seconds.

Following are examples that illustrate procedures for practicing the invention. These examples should not be construed as limiting. All percentages are by weight and all solvent mixture proportions are by volume unless otherwise noted.

### EXAMPLE 1

### Cell Lines and Buffers

CCRF-CEM (CCL-119, T-cell lines, human acute lymphoblastic leukemia), Ramose (CRL-1596, B-cell line, human Burkitt's lymphoma), and Toledo (CRL-2631, human diffuse large cell lymphoma), were obtained from ATCC (American Type Culture Collection) and were cultured in RPMI 1640 medium (ATCC) supplemented with 10% fetal bovie serum (FBS) (heat activated, GIBCO) and 100 IU/mL penicillin-Streptomycin (Cellgro). Cells were washed before and after incubation with wash buffer (4.5 g/L glucose and 5 mM MgCl₂ in Dulbecco's phosphate buffered saline with calcium chloride and magnesium chloride (Sigma)). Binding buffer used for selected was prepared by adding yeast tRNA (0.1 mg/mL) (Sigma) and BSA (1 mg/mL) (Fisher) into wash buffer to reduce background binding. Antibodies against CD2, CD3, CD4, CD5, CD7, and CD45 were purchased from BD Biosciences.

### SELEX Library and Primers

HPLC purified library contained a central randomized sequence of 52 nucleotides (nt) flanked by 18-nt primer hybridization sites (5′-ATA CCA GCT TAT TCA ATT-52-nt-AGA TAG TAA GTG CAA TCT-3′) (SEQ ID NO. 1). A fluorescein isothiocyanate (FITC)-labeled 5′-primer (5′-FITC-ATA CCA GCT TAT TCA ATT-3′) (SEQ ID NO:13) or a tetramethylrhodamine (TMRA)-labeled 5′-primer (5′-TMR-ATA CCA GCT TAT TCA ATT-3′) (SEQ ID NO:14); and a triple biotinylated (trB) 3′-primer (5′-trB-AGA TTG CAC TTA CTA TCT-3′) (SEQ ID NO:15) were used in the PCR reactions for the synthesis of double-labeled, double-stranded DNA molecules. After denaturing in alkaline condition (0.2 M NaOH), the FITC-conjugated sense ssDNA aptamer is separated from the biotinylated anti-sense ssDNA strand by streptavidin-coated sepharose beads (Amersham Bioscience) and used for next round selection. The selection process was monitored using flow cytometry.

### Cell-SELEX Procedure

In accordance with the subject invention, a cell-SELEX process was used to identify and isolate aptamers of interest. An ssDNA pool (200 pmol) dissolved in 400 μL binding buffer was denatured by heating at 95° C. for 5 minutes and cooled on ice for 10 minutes before binding. The ssDNA pool was then incubated with 1−2×10⁶ CCRF-CEM cells (target cells) on ice for one hour. After washing, the bound DNAs were eluted by heating at 95° C. for 5 minutes in 300 μL of binding buffer. The eluted DNAs were then incubated with Ramos cells (negative (control) cells, 5-fold excess than CCRF-CEM cells) for counter-selection on ice for one hour.

After centrifuging, the supernatant was desalted before amplified by PCR using FITC- or biotin-labeled primers (10-20 cycles for 0.5 minutes at 94° C., 0.5 minutes at 46° C., and 0.5 minutes at 72° C., followed by 5 minutes at 72° C.; the Taq-polymerase and dNTP's were obtained from Takala). The selected sense ssDNA is separated from the biotinylated anti-sense ssDNA strand by streptavidin-coated sepharose beads (Amersham Bioscience).

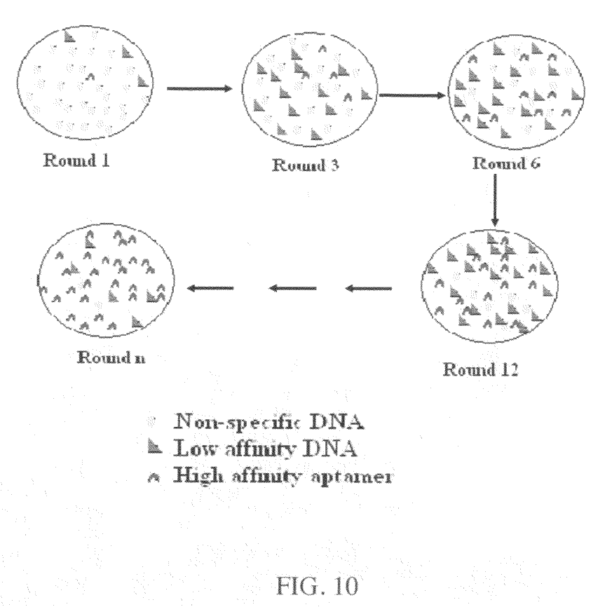

In the first round of selection, 10 nmol of initial ssDNA pool was dissolved in 1 mL binding buffer; and the counter selection group was eliminated. In order to acquire aptamers with high affinity and specificity, the wash strength was enhanced gradually by extending wash time (from 1 minute to 10 minutes), increasing the volume of wash buffer (from 0.5 mL to 5 mL) and the number of washes (from 3 to 5). Additionally, 20% FBS and 50-300 fold molar excess genomic DNA were added to incubation solution. See, for example, FIG. 10, which provides an illustrative schematic for selecting aptamers with high affinity and specificity for cells. FIG. 11 are graphical results from flow cytometry monitoring of the enrichment of target aptamers using a selection process as described herein.

After 25 rounds of selection, any selected ssDNA pool was PCR-amplified using unmodified primers and cloned into Escherichia coli using the TA cloning kit (Invitrogen). Cloned sequences were determined by Genome Sequencing Services Library at the University of Florida.

### Flow Cytometric Analysis

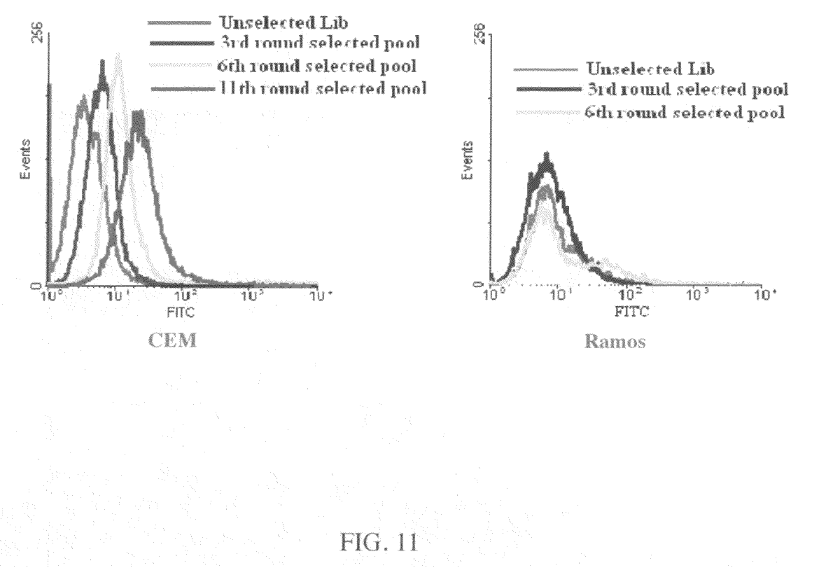

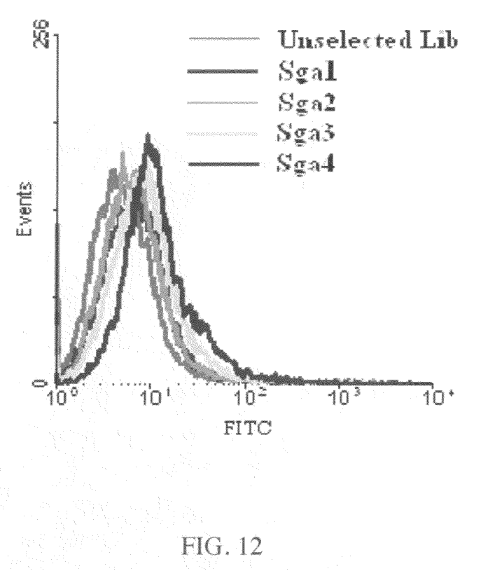

To monitor the enrichment of aptamers after selection, FITC-labeled ssDNA pools were incubated with 1×10⁵ CCRF-CEM cells or Ramos cells, respectively, in 200 μL of binding buffer containing 20% FBS on ice for 50 minutes. Cells were washed twice with 0.7 mL of binding buffer (with 0.1% NaN₂), and suspended in 0.4 mL of binding buffer (with 0.1 NaN₂). The fluorescence was determined with a FACScan cytometer (Becton Dickinson Immunocytometry systems, San Jose, Calif.) by counting 30,000 events. The FITC-labeled unselected ssDNA library was used as negative control. See, for example, FIG. 12, which is a graphical illustration of flow cytometry assay of synthesized DNA-based aptamers using the cell-SELEX method of the invention.

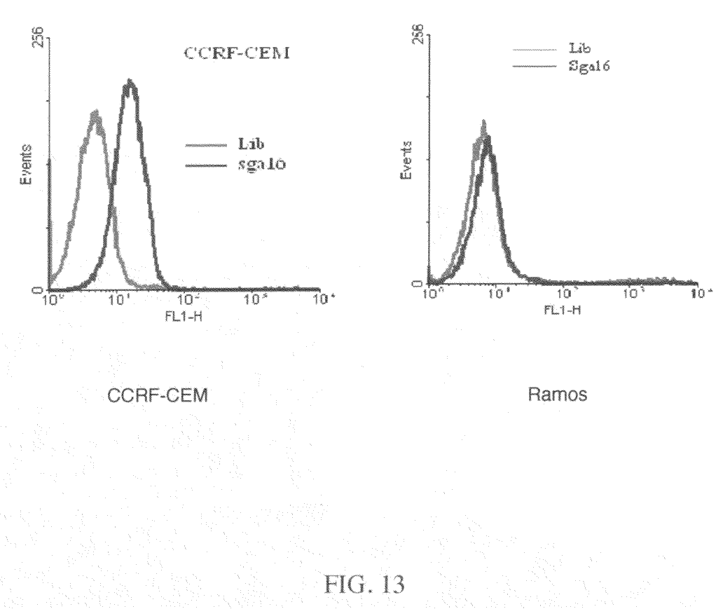

The binding affinity of aptamers was determined by incubating CCRF-CEM cells (5×10⁵) with varying concentrations of FITC-labeled aptamer in 500 μL volume of binding buffer containing 20% FBS on ice for 90 minutes in the dark. Cells were then washed twice with 0.7 mL of the binding buffer with 0.1% sodium azide, suspended in 0.4 mL of binding buffer with 0.1% sodium azide and subjected to flow cytometric analysis within 30 minutes. The FITC-labeled unselected ssDNA library was used as negative control for the nonspecific binding. See, for example, FIG. 13, which illustrates a flow cytometry assay of the binding ability of aptamers as synthesized in accordance with the present invention.

All the experiments for binding assay were repeated 2-4 times. The mean fluorescence intensity of target cells labeled by aptamers was used to calculate for specific binding by subtracting the mean fluorescence intensity of non-specific binding produced by unselected library DNA. The equilibrium dissociation constants (Kd) of the fluorescent ligands were obtained by fitting the dependence of fluorescence intensity of specific binding on the concentration of the ligands to the equation: Y=BmaxX/(Kd+X) using the SigmaPlot software (Jandel Scientific, San Rafael, Calif.).

To test the feasibility of using DNA aptamers for leukemia profiling, FITC labeled aptamers were mixed with PE or PerCP labeled antibodies of CD2, CD3, CD4, CD5, CD7, CD19, and CD45, respectively, and incubated with 2×1 Y=BmaxX/(Kd+X) using the SigmaPlot software (Jandel Scientific, San Rafael, Calif.).

To test the feasibility of using DNA aptamers for leukemia profiling, FITC labeled aptamers were mixed with PE or PerCP labeled antibodies of CD2, CD3, CD4, CD5, CD7, CD19, and CD45, respectively, and incubated with 2×10⁵ CCRF-CEM cells and/or 2×10⁵ cells in human bone marrow aspirates. After performing the washes as described above, the fluorescence was determined with a FACScan cytometer (Becton Dickinson Immunocytometry systems, San Jose, Calif.).

Confocal Imaging of Cell Stained with Aptamer

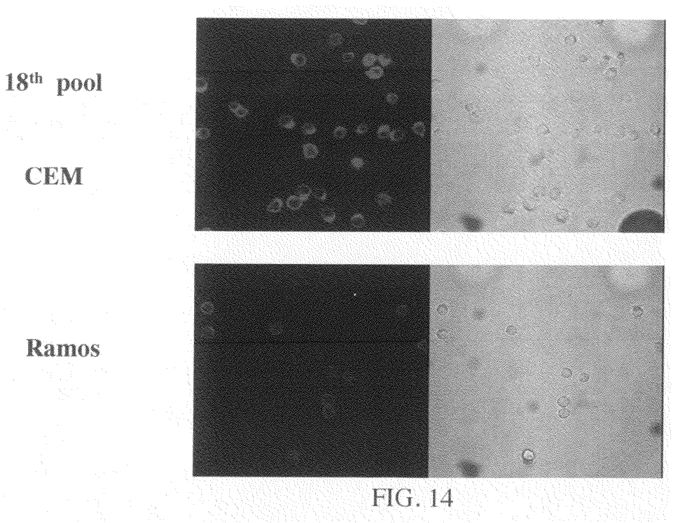

For Confocal imaging, the selected ssDNA pools (or aptamers as selected as described above) were labeled with TMR. Cells incubated with 50 pmol TMR-labeled ssDNA in 100 μL of binding buffer containing 20% FBS on ice for 50 minutes. Other treatment steps were the same as those described in the flow cytometry section. 20 μL of cells suspension bound with TMR-labeled ssDNA were dropped on a thin glass slide placed above a 60× objective on the confocal microscope and covered with a cover slide. The imaging of cells was performed with an Olympus FV500-IX81 confocal microscope (Olympus America Inc., Melville, N.Y.). A 5 mW 543 nM He—Ne laser was the excitation for TAMRA throughout the experiments. The objective used for imaging was a PLAPO60XO3PH 60× oil immersion objective with a numberical aperture of 1.40 from Olympus (Melville, N.Y.). An example of tumor cell imaging with selected aptamer candidates of the invention is illustrated in FIG. 14.

The subject invention provides a cell-SELEX approach for identifying and isolating tumor-specific aptamers that are extremely useful in molecular profiling of targeted or diseased cells. Such a selection does not require prior knowledge of biomarker targets. The selection process is simple, reproducible, and straightforward. The aptamers of the invention can bind to target cells with Kd in the nM to pM range. Using the selected aptamers of the invention as molecular profilers for molecular profiling of cancer cells has yielded interesting information (such as regarding leukemia cells and normal human bone marrow aspirate). For example, some of the subject aptamers can only recognize a subset of the target cells, while others can bind to only one or two types of cancer cells. In addition, the isolation and identification of the target molecules recognized by these selected aptamers provide an effective and rapid way to discover disease biomarkers.

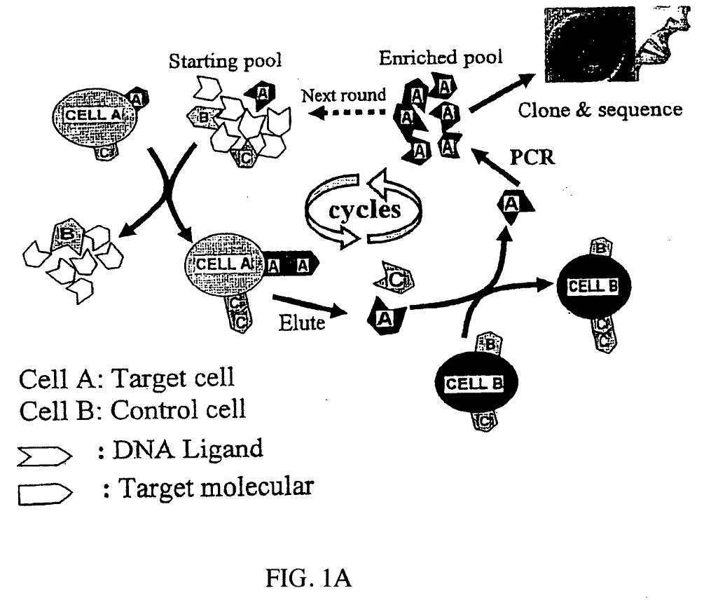

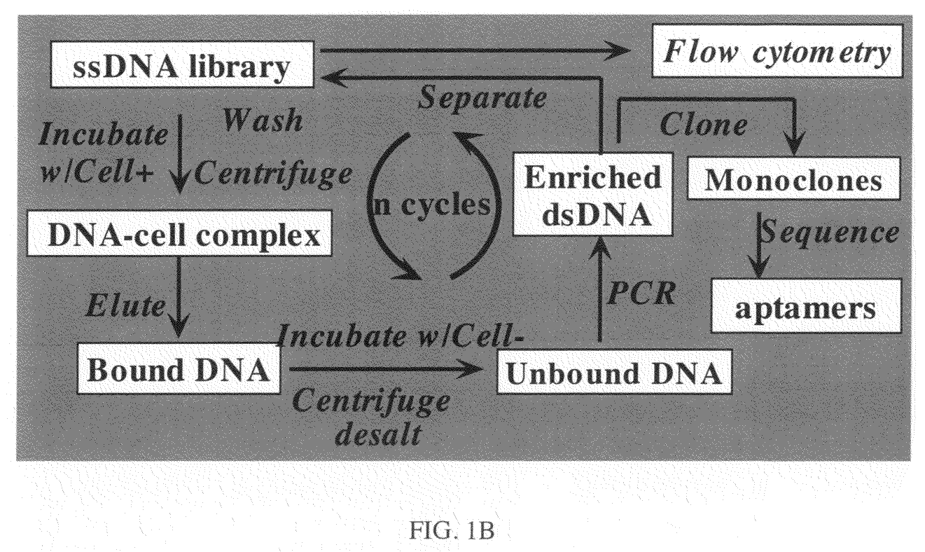

According to the subject invention, the cell-SELEX method can select aptamers that will identify binding entities only expressed by a small subset of target cells (see FIG. 1). In one embodiment, a nucleic acid sample, such as an ssDNA pool, is incubated with CEM cells (target cells). After washing, the bound DNAs are eluted by heating (for example, at 90°). The eluted DNAs are then incubated with control (also referred to as counter-selective) cells (Ramos cells/negative cells) for counter-selection. After centrifuging, the supernatant is collected and the selected DNA is amplified by PCR. The amplified ssDNA are used for the next round of selection (to provide an aptamer with higher affinity for the target biomolecule) or they are cloned and sequenced for aptamer selection. Other forms of nucleic acid samples include, but are not limited to, single-stranded DNA, double-stranded DNA, single-stranded RNA, double-stranded RNA and chemical modifications thereof.

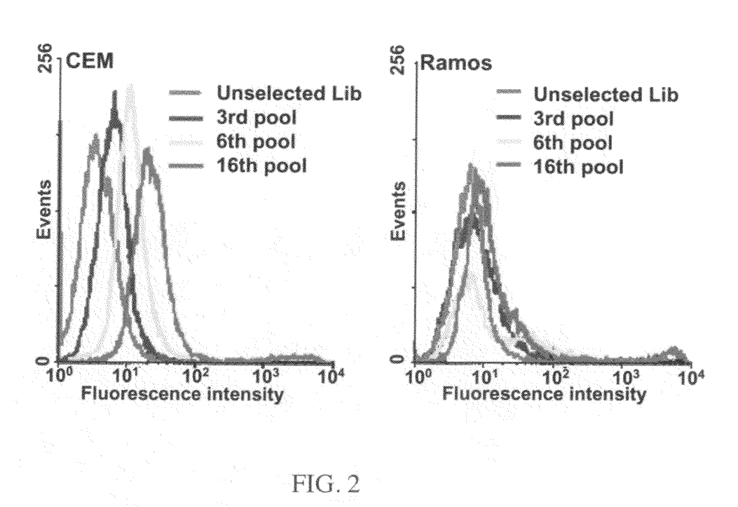

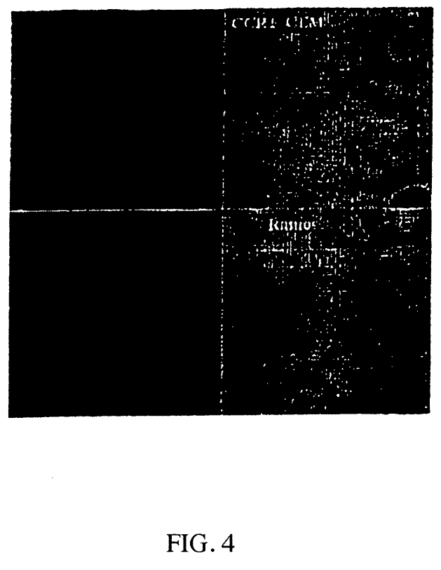

With increased numbers of selection cycles, the DNA sequences with better binding affinity to the target cells were enriched. This was confirmed via observed steady increases in fluorescence intensity in CEM cells (target cells bound with fluorophore-labeled selected DNA sequences) in flow cytometry analysis. There was no significant change in fluorescence intensity on Ramos cells (control cells). These results indicate that DNA probes that specifically recognize surface biomarkers on CEM cells were selected (FIG. 2). The specific binding of the selected pools of DNA probes (aptamers) to the target cells was further confirmed by confocal microscopy imaging (FIG. 4). After incubation with the fluorophore-labeled selected aptamer pool, the CEM cells showed very bright fluorescence on the periphery of cells, while the Ramos cells displayed no significant fluorescence.

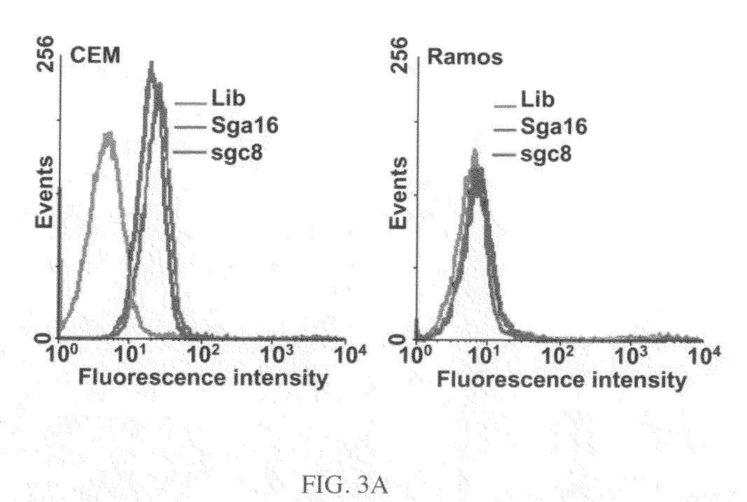

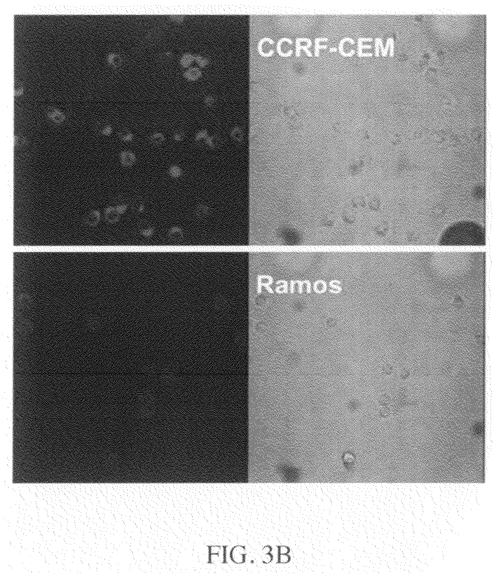

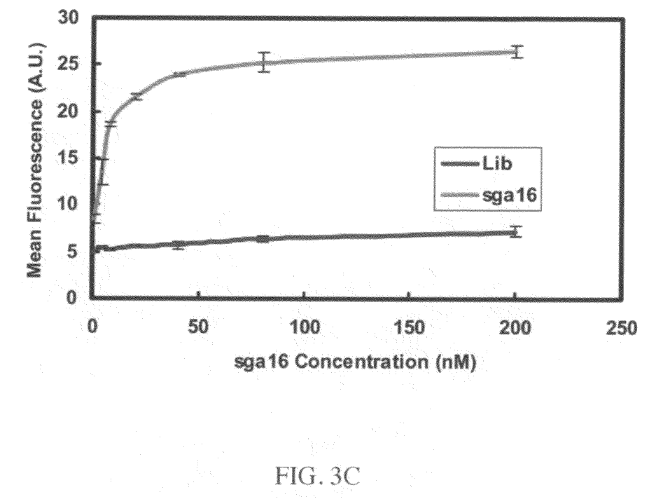

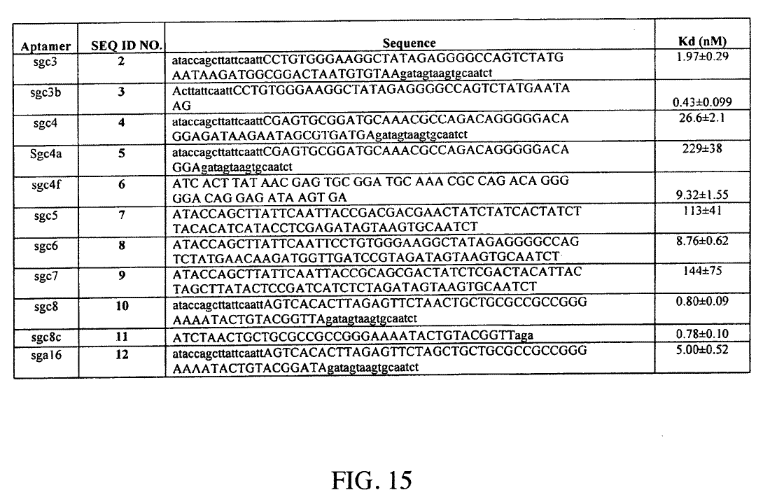

Usually, twenty rounds are necessary to achieve good enrichment of aptamer candidates. The enriched aptamer pools of the invention were cloned and sequenced by high-throughput Genome Sequencing method. Out of 300 clones that were sequenced, eleven sequences were chosen for further characterization, the sequences for each of the probes obtained are listed in FIG. 15. As shown in FIGS. 3A and 3B, homologue aptamers sga16 (SEQ ID NO. 12) and sgc8 (SEQ ID NO. 10) specifically recognize the CEM cells.

FIG. 3A is a flow cytometry assay for the binding of a the FITC-labeled sequence sga16 (SEQ ID NO. 12) and sgc8 (SEQ ID NO. 10) with CEM cells (target cells) and Ramos cells (control cells). The green curve represents the background binding of the unselected DNA library. The blue and pink curves for the two aptamers shift to the right, meaning that the fluorescence in the cells has increased due to more binding of the selected aptamer probes to target biomolecules. The concentration of aptamers in binding buffer was 0.5 μM.

FIG. 3B provides fluorescence confocal images of CEM and Ramose cells stained by TMR-sga16 aptamer, in accordance with the subject invention. The left panel of FIG. 3B is a fluorescence image and the right panel of FIG. 3B is an optical image. The bright image observed with CEM cells indicates a strong binding of sga16 aptamer with the target CEM cells.

FIG. 3C is a graphical illustration of the binding affinity of an FITC-labeled apatmer of the invention to target biomolecules. In particular, FIG. 3C illustrates the binding affinity of FITC-labeled aptamer sequence sga16 to CEM cells. The non-specific binding was measured by using FITC-labeled unselected library DNA. The mean fluorescence intensity of the target cells labeled by the aptamers of the invention was used to calculate for specific binding activity by subtracting the mean fluorescence intensity of non-specific binding produced by unselected library DNA. The equilibrium dissociation constants (Kd) of the fluorescent ligands were obtained by fitting the dependence of fluorescence intensity of the specific binding on the concentration of the ligands to the equation Y=BₘₐₓX/(Kd+X), where X is the concentration of the aptamer probe.

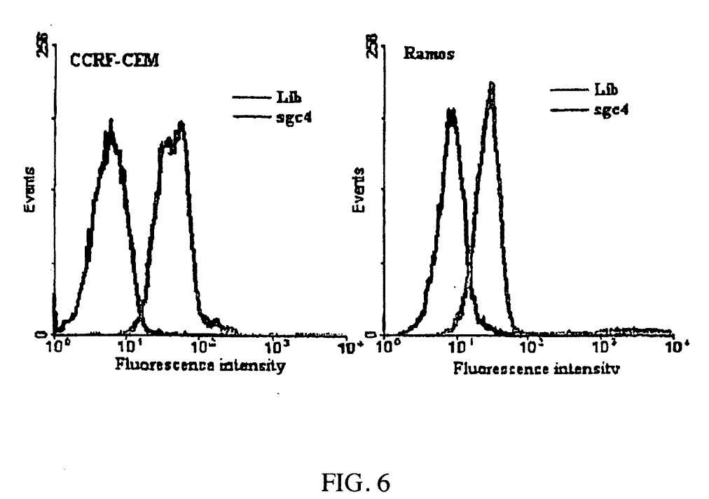

With ten sequences studied, five aptamers were found to have high affinity for CEM cells, sga16 (SEQ ID NO. 12; Kd=5.01±0.52 nmol/L) (FIG. 3C), sgc8 (SEQ ID NO. 10; Kd−0.80±0.09 nmol/L), sgc3 (SEQ ID NO. 2; Kd=1.97±0.3065 nmol/L), sgc6 (Kd=8.76±0.63 nmol/L), and sgc4 (SEQ ID NO. 4; Kd=26.63±2.10 nmol/L). The sgc4 (SEQ ID NO. 4) can also recognize Ramose cells (see FIG. 6), as well as another human B cell lymphoma cell line (Toledo). None of the tested aptamer sequences showed any evidence of competition with antibodies against common antigens such as CD2, CD3, CD4, CD5, CD7, or CD45. This indicates that the aptamers of the subject invention may have surface binding entities that have not been identified yet. The five sequences and their homologues represent approximately 50% of the 300 clones. It is possible that further sequencing of a larger number of clones would produce more aptamers, making molecular profiling of cancer cells feasible, comprehensive, and effective.

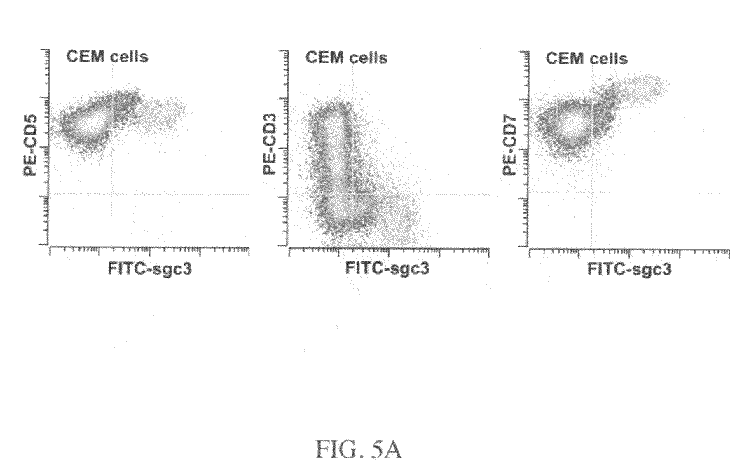

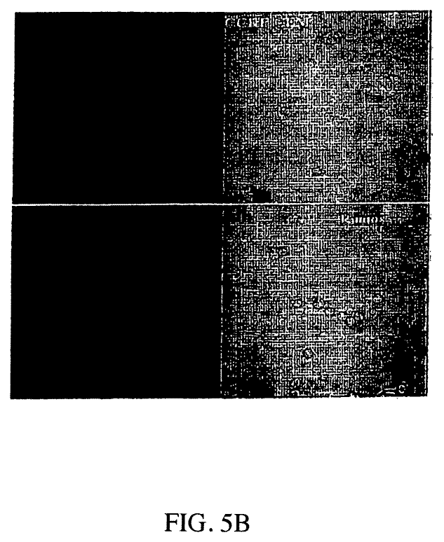

In one embodiment, homologue sequences (such as those illustrated in FIG. 5: sgc3 (SEQ ID NO. 2) and sgc6 (SEQ ID NO. 8)) are identified with the ability to bind to a small subset of the CEM cells with high affinity (20% of the cells). These sgc3 (SEQ ID NO. 2)-labeled cells are viable and express T cell markers, CD5 and CD7, as shown in FIG. 5A. They represent a unique stage of cell differentiation. Specifically, FIG. 5A provides flow cytometry assay images for the binding of aptamer sgc3 (SEQ ID NO. 2) and monoclonal antibodies against CD5, CD7, CD3 on CCRF-CEM cells. The aptamer sgc3 (SEQ ID NO. 2) selectively binds to a subpopulation of CCRF-CEM cells, which express bright CD7 and CD5 but without CD3. The final concentration of sgc3 (SEQ ID NO. 2) in binding buffer was 50 nM.

FIG. 5B are fluorescence confocal images of CEM and Ramos cells stained with TMR-labeled sgc3 (SEQ ID NO. 2). The left panel of FIG. 5B is a fluorescence image and the right panel of FIG. 5B is an optical image. As illustrated in FIG. 5B, there are only a small portion of the CEM cells which are bound with sgc3 (SEQ ID NO. 2).

Accordingly, the subject invention is able to divide presumably same tumor cells into subgroups based on aptamers selected for the same cell line. The excellent specificity by the aptamers of the invention in subset cell recognition will enable highly effective molecular profiling of diseases with minor differences.

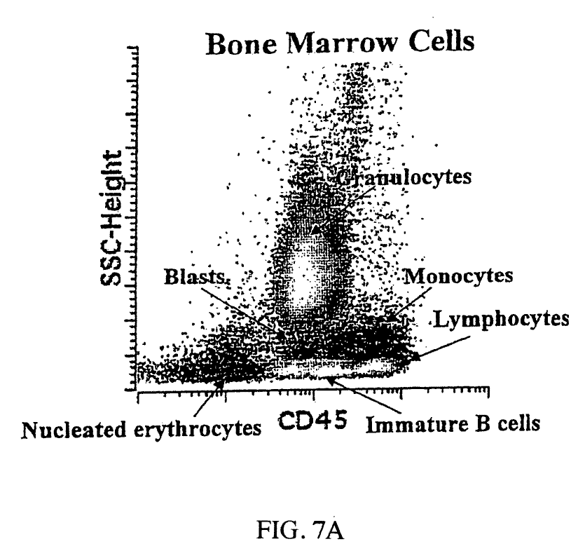

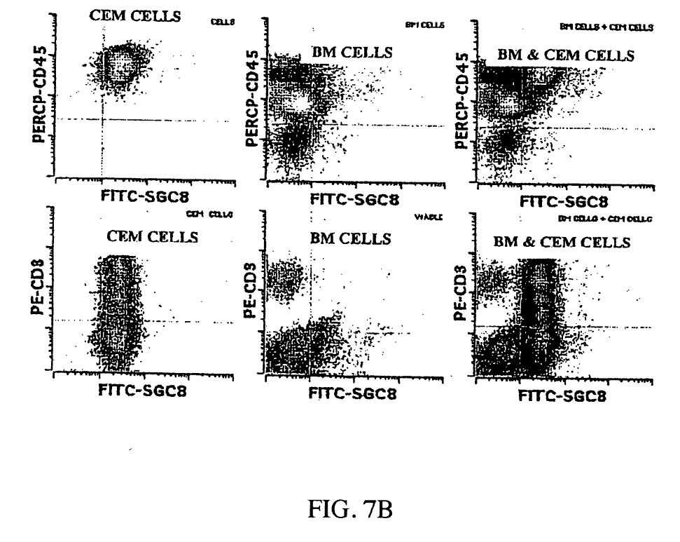

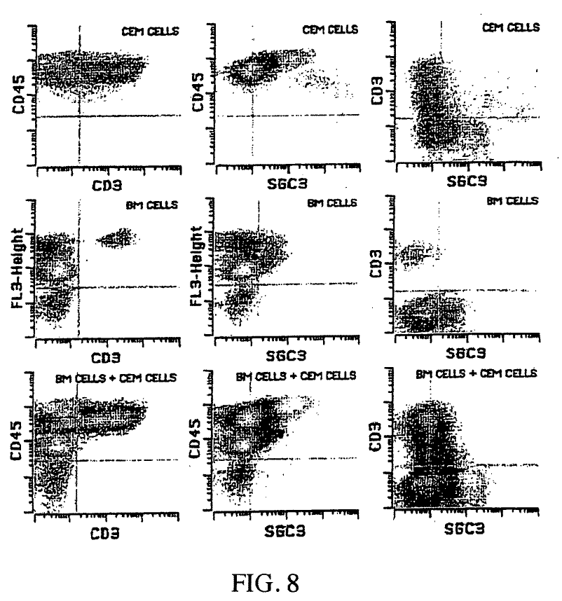

To test the feasibility of the selected aptamers as molecular profilers for molecular profiling, fluorophore-labeled aptamers of the invention and monoclonal antibodies were used to analyze CEM leukemia cells mixed with human bone marrow aspirates. The aspirates consist of mature and immature granulocytes, nucleated erythrocytes, monocytes, T cells, mature and immature B cells (FIG. 7A). Profiling experiments were performed with the aptamers sgc3 (SEQ ID NO. 2), sgc4 (SEQ ID NO. 4), and sgc8 (SEQ ID NO. 10), and the results are summarized in Table 1. Interestingly, the sgc8 (SEQ ID NO. 10) and sgc3 (SEQ ID NO. 2) only recognized cultured leukemia T cells (CEM) and did not bind to normal CD3-positive T cells or any other bone marrow cells (FIG. 7B, FIG. 8). Discrete subpopulations of bone marrow cells (lymphocytes, monocytes, granulocytes, nucleated erthryocytes and early precursors) can be separated by the levels of CD45 expression and side scatter properties (see FIG. 7). The sgc3 (SEQ ID NO. 2) aptamer probe was selected against precursor T acute lymphoblastic leukemia cells (CCRF-CEM) and only recognized a small subset of cultured leukemia cells (CCRF-CEM) (see FIG. 5). Accordingly, FITC-sgc3 can recognize a subset of CCRF-CEM cells mixed with cells from bone marrow aspirates, but did not bind to CD3-positive T cells or other human bone marrow cells.

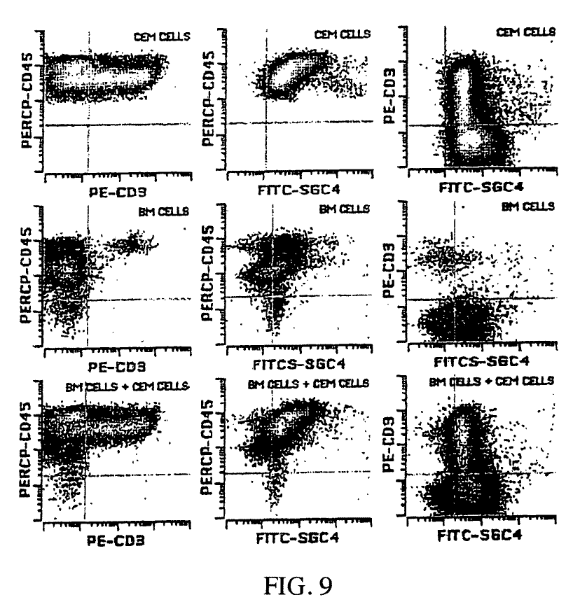

The sgc4 (SEQ ID NO. 4) recognized mature and immature B cells, a subset of CD3-positive T cells, and nucleated erythrocytes from the human bone marrow, and cultured leukemia T cells (CEM) (FIG. 9). The sgc4 (SEQ ID NO. 4) aptamer probe was able to recognize both CCRF-CEM cells and Ramos large B cell lymphoma cells, although it was selected against precursor T acute lymphoblastic leukemia cells (CCRF-CEM). The sgc4 (SEQ ID NO. 4) recognized mature and immature B cells, nucleated erythrocytes, a subset of granulocytes and a small subset of T cells from the human bone marrow aspirates. These results demonstrate that aptamers selected from using the cell-SELEX method described herein is useful for profiling acute leukemia in clinical practice. It is also worth noting that the subject aptamers are selected without prior knowledge of surface markers, which makes the cell-SELEX approach feasible and valuable for cancer biomarker discovery as well as for obtaining molecular signatures of various diseases.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for obtaining a probe specific for extracellular or cell-surface markers comprising:
(a) incubating a sample containing at least one nucleic acid sequence with a sample containing at least one target cell;
(b) allowing substantially all of the target cells to bind with the nucleic acid sequences;
(c) separating and recovering bound nucleic acid sequences to form a first sample;
(d) eluting and incubating the first sample with a sample containing at least one counter-selective cell so that the nucleic acid sequences bind with the counter-selective cells;
(f) separating and recovering unbound nucleic acid sequences to form a second sample; and
(g) cloning and sequencing the nucleic acid sequences of the second sample to obtain a probe specific for the target cell.

2. The method of claim 1, further comprising the steps of:
(f¹) using a quantitative replicative procedure comprising a replicative polymerase reaction following step (f); and
(h) repeating steps (a) through (f¹) at least one more time before proceeding to step (g), wherein the greater number of times step (h) is performed provides a probe with a higher affinity for the target cell.

3. The method of claim 2, further comprising the step of binding a detectable agent to the obtained probe.

4. The method of claim 3, wherein the detectable agent is selected from the group consisting of dansyl; fluorescein; 8-anilino-1-napthalene sulfonate; pyrene; ethenoadenosine; ethidium bromide prollavine monosemicarbazide; p-terphenyl; 2,5-diphenyl-1,3,4-oxadiazole; 2,5-diphenyloxazole; p-bis[2-(5-phenyloxazolyl)]benzene; 1,4-bis-2-(4-methyl-5-phenyloxazolyl)benzene; and lanthanide chelate.

5. The method of claim 3, further comprising the step of monitoring the detectable agent to monitor the affinity of the probe to the target cell.

6. The method of claim 5, wherein flow cytometry is used to monitor the detectable agent.

7. The method of claim 1, wherein the nucleic acid sequence is selected from the group consisting of single-stranded DNA; double-stranded DNA; single-stranded RNA; double-stranded RNA; and chemical modifications thereof.

8. The method of claim 1, wherein the target cell is selected from the group consisting of biological cells.

9. The method of claim 8, wherein the target cell is selected from the group consisting of bacteria; viruses; single-celled protozoan pathogens; cells infected by bacteria, virus, or fungi; and cancer cells.

10. The method of claim 1, wherein the sample containing at least one target cell is selected from the group consisting of animal tissue; biological fluid; environmental substances; plant material; water; beverages; and industrial waste.

11. The method of claim 1, wherein the quantitative replicative procedure is a quantitative polymerase chain reaction.

12. The method of claim 1, wherein separating bound nucleic acid sequences from unbound nucleic acid sequences comprises the step of contacting the sample with an immobilized ligand.

13. The method of claim 12, wherein the ligand is the target cell or the counter-selective cell.

14. The method of claim 12, wherein the immobilized ligand is immobilized on a support matrix selected from the group consisting of resins, beads, magnetic beads, gels, cellulose and silica.

15. The method of claim 13, wherein the support matrix is streptavidin-coated sepharose beads.

16. The method of claim 1, wherein the target cell is a precursor T cell acute lymphoblastic leukemia cell CCRF-CEM and wherein the counter-selective cell is a B cell lymphoma cell line.

17. The method of claim 1, wherein the sample containing at least one nucleic acid sequence comprises a single stranded DNA consisting of 52-mer random DNA sequences flanked by 18-mer primer sequences.

18. The method of claim 17, wherein the sample contains the nucleic acid sequence of SEQ ID NO. 1.

19. The method of claim 1, further comprising the step of incubating a sample comprising the probe with a sample comprising at least one target cell.

20. A probe obtained using the method of claim 1.

21. The probe of claim 20, wherein the probe is selected from the group consisting of: sgc3 (SEQ ID NO. 2); sgc4 (SEQ ID NO. 4); sgc6 (SEQ ID NO. 8); sgc8 (SEQ ID NO. 10); and sga16 (SEQ ID NO. 12).
